(19) United States
(12) Patent Application Publication
(10) Pub. No.: US 2003/0162191 A1
(43) Pub. Date: Aug. 28, 2003

(54) Enzymes and uses relating thereto

(76) Inventors: Eberhard Amtmann, Heidelberg (DE); Katherini Chlichlia, Heidelberg (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/220,905

(22) PCT Filed: Mar. 6, 2001
(86) PCT No.: PCT/GB01/00998

(30) Foreign Application Priority Data
Mar. 6, 2000 (GB) 0005326.4

(51) Int. Cl. C12N 9/16; C12Q 1/68; G01N 33/53; C07K 16/40; A61K 38/17; C12P 21/02; C12N 5/06

(52) U.S. Cl. 435/6; 435/69.1; 435/196; 435/320.1; 435/325; 530/388.26; 435/7.1; 514/12

(57) ABSTRACT

Two novel sphingomyelinases are provided, having optimum activities at pH7 and pH8, respectively. The two proteins have different tissue sources, the pH7 form from e.g. T cell lymphomas, and the pH8 form from e.g. B cell lymphomas. Methods of treatment and diagnosis, fragments, antibodies, nucleic acids and pharmaceutical compositions are also provided.

The present invention relates to sphingomyelinase enzymes (“SMases”), and to nucleic acids encoding them, and to uses of the enzymes and nucleic acids.

There are known to be two forms of sphingomyelinase enzyme, neutral SMase and acid SMase (Gatt, S., Biochem. Biophys. Res. Commun. 68, 235-241, 1976). Neutral SMase is also referred to as “nSMase” and acid SMase is also referred to as “aSMase” or “sSMase”

EP-A-0918750 discloses that guanidine derivatives (as specified in claims 47-50 herein) are inhibitors of sphingomyelin lysis by mouse brain membrane fractions containing SMase enzymes. Greater inhibition is shown of neutral SMase than acid SMase.

The nucleic acid sequence and deduced amino acid sequence of a putative human acid SMase-like phosphodiesterase has been disclosed as EMBL accession number Y08134.

Different nucleic acid sequences and deduced amino acid sequences of nSMase have also been proposed, for example by Chaterjee (WO98/28445 and Chaterjee S et al. J Biol Chem 274, 37407-37412) and by Stoffel et al. (WO99/07855 and Stoffel et al. Chemistry of Physics of Lipids 102, 107-121). The Chaterjee nSMase is found at highest levels in liver and pancreas; the Stoffel nSMase is found at highest levels in kidney and intestine.

The inventors have made the surprising discovery of two distinct and novel forms of neutral SMase. These two forms have different histological distributions and different properties. One form, for which the nucleic acid and amino acid sequences are newly provided herein, is most active at pH7 and hence is herein called “pH7 nSMase”. The other is most active at pH8 and hence is called “pH8 nSMase” or “alkaline nSMase”. The novel pH7 nSMase is present in most tissues tested, whereas pH8 nSMase is absent from many solid tumours, T cell lymphomas and activated or autoreactive T cells, though unlike the Stoffel and Chaterjee nSMases (and like the novel pH7 nSMase), it is expressed at high levels in brain. The pH8 nSMase is also distinguished from the Chaterjee and Stoffel by having a pH optimum at pH8, rather than pH7 or 7.5.

The pH7 nSMase has a deduced amino acid sequence different from those for the Chaterjee and Stoffel nSMases, as disclosed in WO98/28445 and WO99/07855.

Sources of pH7 nSMase are for example human T cell lymphomas, such as Jurkat cells; sources of pH8 nSMase are for example human B cell lymphomas, such as KE 37 or Raji cells.

The newly discovered pH7 nSMase nucleic acid sequence is shown in FIG. 1 (the coding sequence being residues 7 to 1485 or 1371) and the 493 or 455 residue deduced amino acid sequence is shown in FIG. 2.

The inventors have also discovered that 1-(undecylidene-amino)guanidine (C11AG), the exemplified SMase inhibitor of EP-A-0918750, is 200-1000 fold more active on pH7 nSMase than on pH8 nSMase.

EP-A-0918750 disclosed that the sphingomyelinase inhibitors taught therein have advantageous antimicrobial, antiviral, anti-inflammatory (e.g. antishock) effects and effects which influence the growth of cells, e.g. tumour cells.

The inventors have also found that cells which have both pH7 nSMase and pH8 nSMase show resistance to C11AG. However, cells lacking pH8 nSMase (the less sensitive nSMase), such as solid tumour cells, T cell lymphoma cells and activated or autoreactive T cells, are sensitive to the inhibitor. Thus the inhibitors of EP-A-0918750, and other pH7 nSMase antagonists disclosed herein (such as anti-pH7 nSMase antibodies and antisense vectors), may be used selectively against cells identified as pH8 nSMase-deficient, in particular to treat conditions alleviated by sphingomyelinase inhibition, particularly conditions associated with overexpression of sphingomyelinase. Numerous such conditions were identified in EP-A-0918750 (and are recited herein in appendix 1). Preferred conditions are auto-immune disease, inflammatory disease and tumours, particularly solid tumours.

The novel nSMases disclosed herein are thought to be involved in signal transduction. In particular, the inhibition of the pH7 form is capable of specifically affecting CD95-ligand/fas mediated apoptosis, e.g. to increase apoptosis caused by anti-CD95 and/or anti-fas antibodies. Apoptosis can be reduced or resisted by cells expressing the pH8 form of the enzyme. Preferred uses of nSMase inhibitors are therefore to selectively induce or increase apoptosis in cells via this pathway, particularly in cells lacking the pH8 form, for example in the treatment of the conditions listed above.

Accordingly, in a first aspect, the present invention provides an isolated polypeptide comprising a polypeptide having the amino acid sequence set out in FIG. 2.

FIG. 2 shows the deduced amino acid sequence of pH7 nSMase. Position 456 in encoded by a TGA codon in the nucleic acid sequence of FIG. 1, TGA being a stop codon. However, it is believed that readthrough may occur at this codon, so the polypeptide of the invention may be a 455 amino acid sequence or a 493 amino acid sequence.

In a further aspect, the present invention provides an isolated polypeptide including a polypeptide sequence which has at least 80% amino acid sequence identity with the 455 amino acid sequence set out in FIG. 2, and which is identical to FIG. 2 at positions corresponding to one or more of positions 335, 375, 376, 442 and 452-455.

In a further aspect, the present invention provides an isolated polypeptide including a polypeptide sequence which has at least 80% amino acid sequence identity with the 493 amino acid sequence set out in FIG. 2, and which is identical to FIG. 2 at positions corresponding to one or more of positions 335, 375, 376, 442, 452-464, 468-482, 484-486, 489-491 and 493.

The listed positions are those at which the sequence of FIG. 2 differs from the deduced amino acid sequence of deposit Y08134. In relation to polypeptides including a sequence having at least 80% identity with the shorter sequence of FIG. 2, the polypeptide is preferably identical to FIG. 2 at 2 or more, 3 or more, 4 or more, 5 or more, 6 or more, 7 or more or at all 8 said positions. In relation to polypeptides including a sequence having at least 80% identity with the longer sequence of FIG. 2, the polypeptide is preferably identical to FIG. 2 at 2 or more, 4 or more, 6 or more, 10 or more, 15 or more, 20 or more or most preferably at 30 or more said positions.

Preferably the polypeptides of the invention display SMase activity, as defined elsewhere herein. The activity is preferably greater at pH7 than at pH8, more preferably greatest at or about pH7.

In a further aspect, the present invention provides a fragment of a polypeptide as defined above, wherein the fragment has at least 5 amino acids and is identical to FIG. 2 at positions corresponding to one or more of the positions listed above. Preferably the fragment shows at least 80% sequence identity with a portion of corresponding length of the amino acid sequence of FIG. 2.

Desirably the fragment is of the carboxy end of the polypeptide of FIG. 2, preferably including some or all of residues 452-455 or residues 452-493 of FIG. 2. As this region differs significantly in sequence from corresponding regions of other SMase proteins, it is believed to be responsible at least in part for the difference in susceptibility of different SMase proteins to inhibition by the compounds of EP-A-0918750. These fragments can therefore be used in methods of screening for binding partners of pH7 nSMase, e.g. peptides or antibodies which could act as inhibitors.

More preferred levels of sequence identity with the sequences of FIG. 2 are 85%, 90%, 95%, 98% and 99%.

In a further aspect, the present invention provides a method of identifying inhibitors or binding partners of pH7 nSMase, or of optimising previously identified such inhibitors or binding partners, the method comprising determining whether a candidate inhibitor/binding partner possesses the ability, or improved ability relative to a reference inhibitor/binding partner, to inhibit sphingomyelinase activity of, or to bind to, a polypeptide or fragment as defined above. This may be done by for example determining inhibition of or binding to the polypeptide in a cellular environment, e.g. in a cell line expressing the polypeptide, e.g. a transgenic cell line.

Candidate inhibitors may be assayed using the sphingomyelinase inhibition assays disclosed herein and in EP-A-0918750. Optimisation may occur by testing candidate variants of a known inhibitor for improved inhibition.

Binding may be assayed by any convenient means, for example by labelling the polypeptide/fragment and determining the presence of label bound to an immobilised candidate molecule.

Candidate variants may be produced by any convenient means, for example by amino acid substitution, deletion or addition in peptides, or modification, substitution, removal or addition of functional or substituent groups in other organic chemical compounds.

In a further aspect, the present invention provides the use of a novel or optimised inhibitor thus identified for the treatment of, or for the preparation of a medicament for the treatment of, a condition alleviated by nSMase (especially pH7 nSMase) inhibition. Such conditions may be associated with sphingomyelinase overexpression. Suitable conditions for treatment in this way are listed in EP-A-0918750. Preferred conditions are auto-immune disorders (especially arthritis, multiple sclerosis and colitis), inflammatory disease, lymphomas and tumours (particularly solid tumours).

The inhibitor may be used alone or, particularly for the treatment of tumours, may be used as an adjunct in combination with another pharmaceutical. It has been found for example that nSMase inhibitors enhance the anti-tumour efficacy of cisplatin and sensitise tumours which are resistant to cisplatin.

In a further aspect, the present invention provides isolated nucleic acid molecules encoding any one of the above polypeptides or fragments. An example of such a nucleic acid sequence is a nucleic acid sequence comprising nucleotides 7 to 1371 or 1485 as set out in FIG. 1.

The present invention also includes nucleic acid molecules comprising a nucleic acid sequence which has at least 60% sequence identity with nucleotides 7 to 1371 or 1485 of FIG. 1, or a portion thereof of corresponding length, and which encodes the same amino acid as the sequence of FIG. 1 at one or more codons corresponding to codons 335, 375, 376, 442, 452-464, 468-482, 484-486, 489-491 and 493 of FIG. 1 (i.e. those codons of FIG. 1 which encode amino acids which differ from those at corresponding positions in known SMases, codon 1 corresponding to nucleotides 7 to 9). Preferred nucleic acids are those comprising a nucleic acid sequence having at least 60% identity with the portion of the nucleic acid sequence of FIG. 1 which encodes the 455 or 493 amino acid sequences of FIG. 2.

In relation to nucleic acid molecules comprising a nucleic acid sequence having at least 60% identity with the shorter portion, said nucleic acid sequence preferably encodes the same amino acid as the nucleic acid of FIG. 1 at 2 or more, 3 or more, 4 or more, 5 or more, 6 or more, 7 or more or at all 8 said positions. In relation to nucleic acid molecules comprising a nucleic acid sequence having at least 60% identity with the longer sequence, said nucleic acid sequence preferably encodes the same amino acid as the nucleic acid of FIG. 1 at 2 or more, 4 or more, 6 or more, 10 or more, 15 or more, 20 or more or most preferably at 30 or more said positions.

The nucleic acid sequence is preferably identical to the nucleic acid sequence of FIG. 1 at one or more residues corresponding to positions 570, 1008, 1009, 1131, 1132, 1326, 1330, 1353 and 1360. These are positions at which the sequence of FIG. 1 differs from the sequence of deposit Y08134. Preferably the nucleic acid lacks an insert either between residues corresponding to positions 1362 and 1363 and/or between residues corresponding to positions 1389 and 1390. Inserts between these positions in the Y08134 nucleic acid sequence lead to frame shifts relative to the sequence of FIG. 1.

Preferably the nucleic acid molecules encode polypeptides having SMase activity, which activity is preferably greater at pH7 than at pH8, more preferably greatest at or about pH7.

Preferred sequence identities for the nucleic acids of the invention are 70%, 80%, 85%, 90%, 95%, 98% and 99%.

In further aspects, the present invention provides an expression vector comprising any one of the above nucleic acid sequences operably linked to control sequences to direct its expression, and a host cell transformed with the vector. The present invention also includes a method of producing polypeptides comprising culturing the host cell and isolating the polypeptide or fragment thus produced.

Such host cells may find use in screening for agonists or antagonists of pH7 nSMase.

In a further aspect, the present invention provides an expression vector as defined above for use in methods of medical treatment, e.g. gene therapy.

In a further aspect, the present invention provides a cell line for transplantation into a patient, the cell line being transformed with and capable of expressing nucleic acid as defined above. In one embodiment, the cell lines can be encapsulated, e.g. in a biocompatible polymer so that the polypeptide produced by the cell line can be secreted into the patient, while preventing rejection by the immune system of the host. Methods for encapsulating cells in biocompatible polymers are described in WO93/16687 and WO96/31199.

In further aspects, the present invention provides a nucleic acid sequence complementary to any one of the above nucleic acid sequences; a transcription vector comprising that complementary sequence operably linked to control sequences to direct its transcription (an “antisense vector”), and host cells transformed with the vectors. Transcription of the complementary nucleic acid sequence would lead to the production of antisense mRNA, which would be capable of reducing or preventing normal expression of pH7 nSMase by a cell which transcribes pH7 nSMase mRNA. Further, the present invention provides an antisense vector as defined above for use in methods of gene therapy, e.g. in the treatment of patients who overproduce pH7 nSMase.

In a further aspect, the present invention provides a pharmaceutical composition comprising any of the nucleic acid molecules, vectors or host cells as defined above.

In a further aspect, the present invention provides a pharmaceutical composition comprising one or more polypeptides or fragments as defined above.

In further aspects, the present invention provides the above polypeptides, nucleic acid molecules and vectors for use in methods of medical treatment. The present invention further provides the use of the polypeptides, nucleic acids and expression vectors for the treatment of, and for the preparation of medicaments for the treatment of, conditions associated with insufficient production of SMase (preferably insufficient production of nSMase, more preferably pH7 nSMase). The present invention further provides the use of the complementary nucleic acids and antisense vectors for the treatment of, and for the preparation of medicaments for the treatment of, conditions alleviated by sphingomyelinase inhibition, such as conditions associated with overproduction of SMase (preferably overproduction of nSMase, more preferably pH7 nSMase). Preferably the condition is one also associated with a lack of or reduced pH8 nSMase expression. Preferred conditions are auto-immune disorders (especially arthritis, multiple sclerosis and colitis), inflammatory disease, lymphomas and tumours (particularly solid tumours). The complementary nucleic acid and/or antisense vector may in particular be used as an adjunct in combination with another pharmaceutical for the treatment of tumours.

In a further aspect, the present invention provides antibodies capable of specifically binding to the above polypeptides. These antibodies can be used in assays to detect and quantify the presence of pH7 nSMase, in methods of purifying pH7 nSMase, and in pharmaceutical compositions, e.g. to neutralize or inhibit pH7 nSMase in the conditions mentioned above. Accordingly antagonists, such as antibodies which block the expression of pH7 nSMase or neutralize pH7 nSMase, can be used for treating such conditions and are included within the present invention.

In a further aspect, the present invention provides a method for determining the presence of nucleic acid encoding pH7 nSMase and/or mutations within such nucleic acid in a test sample, the method comprising detecting the hybridization of test sample nucleic acid to a nucleic acid probe based on the nucleic acid sequence set out in FIG. 1. Preferably the probe encodes, or is complementary to a sequence encoding, a fragment as defined above. In particular the probe preferably encodes, or is complementary to a sequence encoding, some or all of amino acid residues 452-455 or 452-493 of FIG. 2.

In a further aspect, the present invention provides the use of nucleic acid as defined above in the design of antisense oligonucleotides to restrict pH7 nSMase expression in a population of cells, i.e. phosphorothiolated or chloresterol linked oligonucleotides which can facilitate internalization and stabilization of the oligonucleotides.

In a further aspect, the present invention provides a method of amplifying a nucleic acid test sample comprising priming a nucleic acid polymerase reaction with nucleic acid encoding a polypeptide or fragment as defined above. The present invention also provides the use of the above nucleic acid in the search for mutations in the pH7 nSMase gene, e.g. using techniques such as single stranded conformation polymorphism (SSCP).

The amplification products and/or mutations in the pH7 nSMase gene may also encode proteins having sphingomyelinase activity as defined elsewhere herein, which activity may be greatest at or about pH7.

The present invention also provides a method of assaying a test sample for susceptibility to SMase inhibition, particularly susceptibility to inhibition by the guanidine derivatives of EP-A-0918750, particularly C11AG, and/or the pH7 nSMase antagonists disclosed herein, the method comprising demonstrating in the test sample reduced expression, low expression or the absence or substantial absence of expression of pH8 nSMase. Preferably the presence of expression of pH7 nSMase is also demonstrated. For this purpose, transcription of mRNA may be taken to be a marker of protein expression, as well as actual protein levels. “Reduced expression” may mean that the expression of pH8 nSMase in the test sample is lower than that in other samples of the cell or tissue type; “low expression” may mean that the expression of pH8 nSMase in the test sample is lower than that of pH7 nSMase.

The present invention also provides a method, having demonstrated the absence of pH8 nSMase expression in a cell or tissue, of selectively inhibiting SMase in said cell or tissue, or in a cell or tissue of the same type, the method comprising administering an inhibitor of pH7 nSMase to said cell or tissue. The inhibitor may for example be an antibody or an antisense vector. Preferably, however, it is a guanidine derivative as disclosed in EP-A-0918750, more preferably C11AG.

The expression of pH8 nSMase may be determined by any convenient means. In particular, the activity of pH7 and pH8 nSMase can be determined separately using assays at different pH, as described herein in the examples. At pH6.5, pH8 nSMase is inactive and pH7 nSMase is active and vice versa at pH8.5.

The present invention also provides pH8 nSMase polypeptide and fragments, e.g. polypeptide as obtainable by or as obtained by isolating nSMase activity from human B cell lymphoma, e.g. KE 37 or Raji cell lines, and fragments thereof. It has been found that nSMase activity in these cell lines is overwhelmingly predominately pH8 nSMase activity.

The term “pH8 nSMase polypeptide” is to be understood to include variants of the naturally occurring sequence obtainable from such cells, such as allelic variants or variants by amino acid insertion, deletion, substitution or modification.

However, the polypeptide may also be isolatable from other cell lines and the invention should not be construed as being limited to isolation from B cell lymphoma.

The invention further provides unique fragments or active portions of the above pH8 nSMase polypeptide, antibodies capable of specifically binding the pH8 nSMase polypeptide or unique fragments or active portions, nucleic acid encoding such polypeptide, fragments and portions (whether obtainable by cloning the naturally occurring pH8 nSMase gene, or other sequences which also encode said polypeptides etc. by virtue of the degeneracy of the genetic code), methods of identifying and/or optimising inhibitors of nSMase by screening for inhibition SMase activity of or binding to said polypeptide, fragments or active portions, and the use of inhibitors for medical treatments, particularly for use on tissues that exhibit pH8 nSMase expression but low, reduced or no pH7 nSMase expresssion. Where applicable, the disclosure herein relating to pH7 nSMase applies also to pH8 nSMase, mutatatis mutandis as appropriate. For example, inhibitors of pH8 nSMase may be used to cause apoptosis in cells which express pH8 nSMase but not pH7 nSMase and/or to treat conditions associated with expression of pH8 nSMase but not pH7 nSMase (e.g. B cell lymphomas).

Inhibitors of and/or antibodies to pH8 nSMase may be specific for pH8 nSMase compared to pH7 nSMase, though it is also contemplated that inhibitors may be found that are active against both forms. Thus for example, an inhibitor of pH8 nSMase may be capable of use in treating diseases associated with expression of pH7 nSMase but not pH8 nSMase (e.g. T cell lymphoma), if the inhibitor is also capable of inhibiting pH7 nSMase.

These and other aspects of the present invention are described in detail below, with reference to the accompanying figures.

### BRIEF DESCRIPTION OF THE FIGURES

FIG. 1 sets out the cDNA sequence of pH7 nSMase enzyme. The coding sequence is represented by nucleotides 7 to 1485 or 1371 of this sequence.

FIG. 2 sets out the 493 or 455 residue deduced amino acid sequence encoded by the pH7 nSMase nucleic acid sequence of FIG. 1.

FIG. 3 shows an alignment between the pH7 nSMase cDNA sequence of FIG. 1 (upper sequence) and the known nucleic acid sequence deposited under GenBank/EMBL accession number Y08134 (lower sequence).

FIG. 4 shows an alignment between the pH7 nSMase deduced amino acid sequence of FIG. 2 (lower sequence) and that of GenBank/EMBL accession number Y08134 (upper sequence).

FIG. 5 shows that two different tissue sources yield nSMase activities having distinct pH dependencies.

FIGS. 5a-c show the activities in pH7 and pH8 nSMase assays of various tissue and organ samples: part a mouse organs, part b lymphomas, part c human tumour cells.

FIG. 6 shows inhibition of pH7 and pH8 nSMase and aSMase activity by C11AG.

FIG. 7 shows that an inhibitor of pH7 nSMase stimulates apoptosis in human T-cell lymphoma (Jurkat) cells.

### DETAILED DESCRIPTION

pH7 nSMase Nucleic Acid

The pH7 nSMase nucleic acid molecules of the present invention include nucleic acid molecules having a nucleotide sequence which encodes a polypeptide including the amino acid sequence shown in FIG. 2.

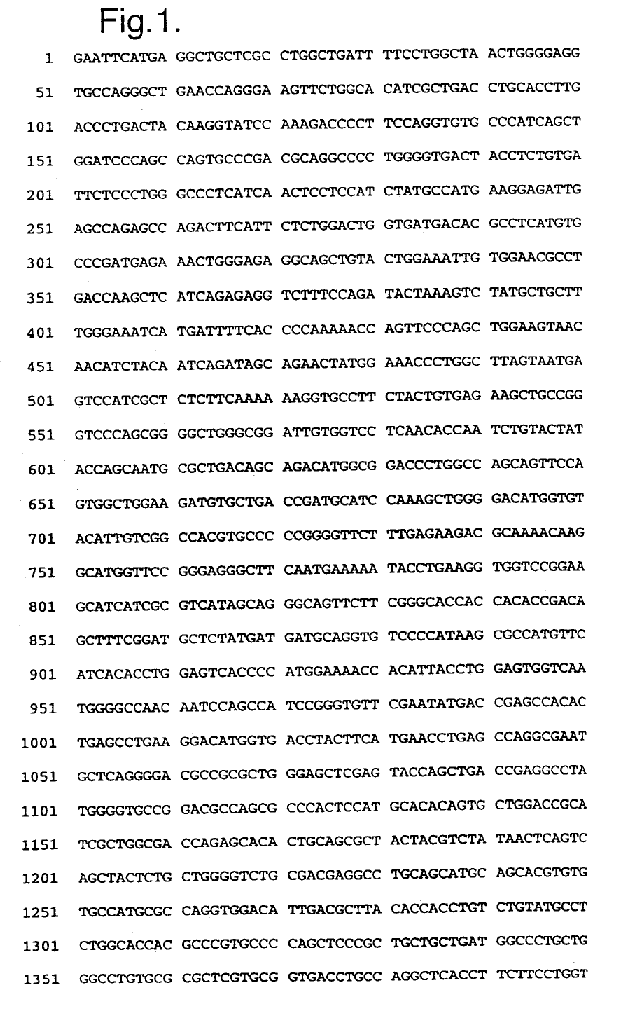

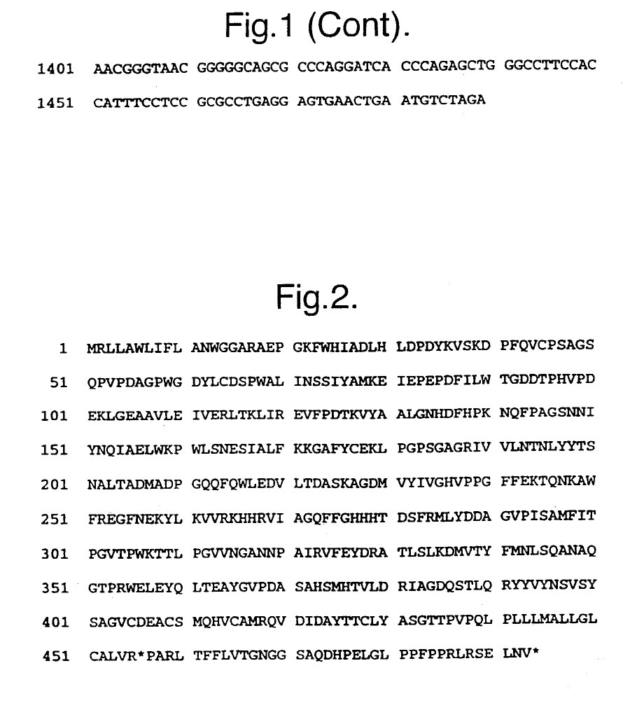

The coding sequence may be that shown in FIG. 1, residues 7 to 1485 or 1371, or a complementary nucleic acid sequence, or it may be a mutant, variant, derivative or allele of these sequences. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in FIG. 1 yet encode a polypeptide with the same amino acid sequence (i.e. the amino acid sequence shown in FIG. 2). The amino acid sequence of the complete pH7 nSMase polypeptide shown in FIG. 2 consists of 493 or 455 amino acids.

On the other hand, the encoded polypeptide may comprise an amino acid sequence which differs by one or more amino acid residues from the amino acid sequence shown in FIG. 2. Nucleic acid encoding a polypeptide which is an amino acid sequence mutant, variant, derivative or allele of the sequence shown in FIG. 1 is further provided by the present invention. Such polypeptides are discussed below.

The present invention also includes fragments of the nucleic acid sequences described herein, preferably fragments of the sequence of FIG. 1, the fragments preferably being at least 15, 18, 21, 24, 30, 45, 60, 120, 150, 200, or 300, or 500 nucleotides in length.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding all or part of the pH7 nSMase gene and/or its regulatory elements can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992). These techniques include (i) the use of the polymerase chain reaction (PCR) to amplify samples of such nucleic acid, e.g. from genomic sources, (ii) chemical synthesis, or (iii) amplification in E. coli.

Modifications to the pH7 nSMase sequences can be made, e.g. using site directed mutagenesis, to provide expression of modified pH7 nSMase polypeptide or to take account of codon preference in the host cells used to express the nucleic acid.

In order to obtain expression of the nucleic acid sequences, the sequences can be incorporated in a vector having control sequences operably linked to the nucleic acid to control its expression. The vectors may include other sequences such as promoters or enhancers to drive the expression of the inserted nucleic acid, nucleic acid sequences so that the pH7 nSMase polypeptide is produced as a fusion and/or nucleic acid encoding secretion signals so that the polypeptide produced in the host cell is secreted from the cell. pH7 nSMase polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the polypeptide expressed in those cells, e.g. controlling where the polypeptide is deposited in the host cells or affecting properties such as its glycosylation and phosphorylation.

PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises steps of denaturation of template nucleic acid (if double-stranded), annealing of primer to target, and polymerisation. The nucleic acid probed or used as template in the amplification reaction may be genomic DNA, cDNA or RNA. PCR can be used to amplify specific sequences from genomic DNA, specific RNA sequences and cDNA transcribed from mRNA, bacteriophage or plasmid sequences. The nucleic acid sequences provided herein readily allow the skilled person to design PCR primers. References for the general use of PCR techniques include Mullis et al, Cold Spring Harbor Symp. Quant. Biol., 51:263, (1987), Ehrlich (ed), PCR Technology, Stockton Press, NY, 1989, Ehrlich et al, Science, 252:1643-1650, (1991), “PCR protocols; A Guide to Methods and Applications”, Eds. Innis et al, Academic Press, New York, (1990).

Also included within the scope of the invention are antisense oligonucleotide sequences based on the pH7 nSMase nucleic acid sequences described herein, particularly to block the synthesis of pH7 nSMase in situations where its overexpression has a deleterious effect, or where it is desirable to inhibit pH7 nSMase, e.g. in the treatment of conditions alleviated by SMase inhibition (for which see EP-A-0918750), particularly in the treatment of autoimmune disorders (especially arthritis, multiple sclerosis and colitis), inflammatory disease, lymphomas (especially T-cell lymphomas) and tumours (particularly solid tumours). Antisense oligonucleotides may be designed to hybridize to the complementary sequence of nucleic acid, pre-mRNA or mature mRNA, interfering with the production of polypeptide encoded by a given DNA sequence (e.g. either native pH7 nSMase polypeptide or a mutant form thereof), so that its expression is reduced or prevented altogether. In addition to the pH7 nSMase coding sequence, antisense techniques can be used to target the control sequences of the pH7 nSMase gene, e.g. in the 5′ flanking sequence of the pH7 nSMase coding sequence, whereby the antisense oligonucleotides can interfere with pH7 nSMase control sequences. The construction of antisense sequences and their use is described in Peyman and Ulman, Chemical Reviews, 90:543-584, (1990), Crooke, Ann. Rev. Pharmacol. Toxicol., 32:329-376, (1992), and Zamecnik and Stephenson, P.N.A.S, 75:280-284, (1974).

The nucleic acid sequence provided in FIG. 1 is useful for identifying nucleic acid of interest (and which may be according to the present invention) in a test sample. The present invention provides a method of obtaining nucleic acid of interest, the method including hybridization of a probe having the sequence of one of the nucleic acid fragments according to the invention, or a sequence complementary thereto, to target nucleic acid.

Hybridization is generally followed by identification of successful hybridization and isolation of nucleic acid which has hybridized to the probe, which may involve one or more steps of PCR.

Nucleic acid according to the present invention is obtainable using one or more oligonucleotide probes or primers designed to hybridize with one or more fragments of the nucleic acid sequence shown in FIG. 1, particularly fragments including codons which encode amino acids which differ between the newly discovered pH7 nSMase and other SMases (i.e. residues 335, 375, 376, 442, 452-464, 468-482, 484-486, 489-491 and 493 of FIG. 2). A primer designed to hybridize with a fragment of the nucleic acid sequence shown in the above figures may be used in conjunction with one or more oligonucleotides designed to hybridize to a sequence in a cloning vector within which target nucleic acid has been cloned, or in so-called “RACE” (rapid amplification of cDNA ends) in which cDNAs in a library are ligated to an oligonucleotide linker and PCR is performed using a primer which hybridizes with the sequence shown in FIG. 1 and a primer which hybridizes to the oligonucleotide linker.

Such oligonucleotide probes or primers, as well as the full-length sequence (and mutants, alleles, variants and derivatives) are also useful in screening a test sample containing nucleic acid for the presence of alleles, mutants and variants, especially those that lead to the production of inactive forms of pH7 nSMase protein, the probes hybridizing with a target sequence from a sample obtained from the individual being tested. The conditions of the hybridization can be controlled to minimise non-specific binding, and stringent to moderately stringent hybridization conditions are preferred. The skilled person is readily able to design such probes, label them and devise suitable conditions for the hybridization reactions, assisted by textbooks such as Sambrook et al (1989) and Ausubel et al (1992).

As well as determining the presence of polymorphisms or mutations in the pH7 nSMase sequence, the probes may also be used to determine whether mRNA encoding pH7 nSMase is present in a cell or tissue.

Nucleic acid isolated and/or purified from one or more cells (e.g. human) or a nucleic acid library derived from nucleic acid isolated and/or purified from cells (e.g. a cDNA library derived from mRNA isolated from the cells), may be probed under conditions for selective hybridization and/or subjected to a specific nucleic acid amplification reaction such as the polymerase chain reaction (PCR). Polymorphisms within this gene may be used as markers for human genetic diseases such as those mentioned above.

In the context of cloning, it may be necessary for one or more gene fragments to be ligated to generate a full-length coding sequence. Also, where a full-length encoding nucleic acid molecule has not been obtained, a smaller molecule representing part of the full molecule, may be used to obtain full-length clones. Inserts may be prepared from partial cDNA clones and used to screen cDNA libraries. The full-length clones isolated may be subcloned into expression vectors and activity assayed by transfection into suitable host cells, e.g. with a reporter plasmid.

A method may include hybridization of one or more (e.g. two) probes or primers to target nucleic acid. Where the nucleic acid is double-stranded DNA, hybridization will generally be preceded by denaturation to produce single-stranded DNA. The hybridization may be as part of a PCR procedure, or as part of a probing procedure not involving PCR. An example procedure would be a combination of PCR and low stringency hybridization. A screening procedure, chosen from the many available to those skilled in the art, is used to identify successful hybridization events and isolated hybridized nucleic acid.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RNAse cleavage and allele specific oligonucleotide probing.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridized to the DNA fragments on the filter and binding determined. DNA for probing may be prepared from RNA preparations from cells.

Preliminary experiments may be performed by hybridizing under low stringency conditions various probes to Southern blots of DNA digested with restriction enzymes. Suitable conditions would be achieved when a large number of hybridizing fragments were obtained while the background hybridization was low. Using these conditions, nucleic acid libraries, e.g. cDNA libraries representative of expressed sequences, may be searched.

Those skilled in the art are well able to employ suitable conditions of the desired stringency for selective hybridization, taking into account factors such as oligonucleotide length and base composition, temperature and so on.

On the basis of amino acid sequence information, oligonucleotide probes or primers may be designed, taking into account the degeneracy of the genetic code, and where appropriate, codon usage of the organism from the candidate nucleic acid is derived. An oligonucleotide for use in nucleic acid amplification may have about 10 or fewer codons (e.g. 6, 7 or 8), i.e. be about 30 or fewer nucleotides in length (e.g. 18, 21 or 24). Generally specific primers are upwards of 14 nucleotides in length, but not more than 18-20. Those skilled in the art are well versed in the design of primers for use processes such as PCR.

A further aspect of the present invention provides an oligonucleotide or polynucleotide fragment of the nucleotide sequence shown in FIG. 1, or a complementary sequence, in particular for use in a method of obtaining and/or screening nucleic acid. The sequences referred to above may be modified by addition, substitution, insertion or deletion of one or more nucleotides, but preferably without abolition of ability to hybridize selectively with nucleic acid with the sequence shown in FIG. 1, that is wherein the degree of identity of the oligonucleotide or polynucleotide with one of the sequences given is sufficiently high.

In some preferred embodiments, oligonucleotides according to the present invention that are fragments of the sequence shown in FIG. 1, or an allele thereof, are at least 10 nucleotides in length, more preferably at least 15 nucleotides in length, more preferably at least 20 nucleotides in length, more preferably at least 40 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of a sequence indicative of susceptibility to one of the conditions mentioned above.

Nucleic acid according to the present invention may be used in methods of gene therapy, for instance in treatment of individuals with the aim of preventing or curing (wholly or partially) the above mentioned conditions. This too is discussed below.

A convenient way of producing a polypeptide according to the present invention is to express nucleic acid encoding it, by use of the nucleic acid in an expression system. The use of expression systems has reached an advanced degree of sophistication.

Accordingly, the present invention also encompasses a method of making a polypeptide (as disclosed), the method including expression from nucleic acid encoding the polypeptide (generally nucleic acid according to the invention). This may conveniently be achieved by growing a host cell in culture, containing such a vector, under appropriate conditions which cause or allow expression of the polypeptide. Polypeptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Thus, a further aspect of the present invention provides a host cell containing nucleic acid as disclosed herein. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

A still further aspect provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed. Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

Introduction of nucleic acid may take place in vivo by way of gene therapy, as discussed below.

A host cell containing nucleic acid according to the present invention, e.g. as a result of introduction of the nucleic acid into the cell or into an ancestor of the cell and/or genetic alteration of the sequence endogenous to the cell or ancestor (which introduction or alteration may take place in vivo or ex vivo), may be comprised (e.g. in the soma) within an organism which is an animal, particularly a mammal, which may be human or non-human, such as rabbit, guinea pig, rat, mouse or other rodent, cat, dog, pig, sheep, goat, cattle or horse, or which is a bird, such as a chicken. Genetically modified or transgenic animals or birds comprising such a cell are also provided as further aspects of the present invention.

This may have a therapeutic aim. (Gene therapy is discussed below.) The presence of a mutant, allele or variant sequence within cells of an organism, particularly when in place of a homologous endogenous sequence, may allow the organism to be used as a model in testing and/or studying the role of the pH7 nSMase gene or substances which modulate activity of the encoded nSMase polypeptide in vitro.

Instead of or as well as being used for the production of a polypeptide encoded by a transgene, host cells may be used as a nucleic acid factory to replicate the nucleic acid of interest in order to generate large amounts of it. Multiple copies of nucleic acid of interest may be made within a cell when coupled to an amplifiable gene such as DHFR. Host cells transformed with nucleic acid of interest, or which are descended from host cells into which nucleic acid was introduced, may be cultured under suitable conditions, e.g. in a fermenter, taken from the culture and subjected to processing to purify the nucleic acid. Following purification, the nucleic acid or one or more fragments thereof may be used as desired, for instance in a diagnostic or prognostic assay as discussed elsewhere herein.

pH7 nSMase Proteins

The skilled person can use the techniques described herein and others well known in the art to produce large amounts of the pH7 nSMase polypeptide, or fragments or active portions thereof, for use as pharmaceuticals, in the developments of drugs and for further study into its properties and role in vivo.

Thus, a further aspect of the present invention provides a polypeptide which has the amino acid sequence shown in FIG. 2, which may be in isolated and/or purified form, free or substantially free of material with which it is naturally associated, such as other polypeptides or such as human polypeptides other than pH7 nSMase polypeptide or (for example if produced by expression in a prokaryotic cell) lacking in native glycosylation, e.g. unglycosylated. In particular, the polypeptide may be free or substantially free of other SMase enzymes, particularly pH8 nSMase.

Polypeptides which are amino acid sequence variants, alleles, derivatives or mutants are also provided by the present invention. A polypeptide which is a variant, allele, derivative or mutant may have an amino acid sequence which differs from that given in FIG. 2 by one or more of addition, substitution, deletion and insertion of one or more amino acids. Preferred polypeptides have SMase function, that is to say the ability to lyse sphingomyelin, thereby to release phosphorylcholine, for example as assayed in the sphingomyelinase inhibition assays described herein or in EP-A-0918750.

A polypeptide which is an amino acid sequence variant, allele, derivative or mutant of the amino acid sequence shown in FIG. 2 may comprise an amino acid sequence which shares greater than about 80%, greater than about 90%, greater than about 95%, greater than about 97%, greater than about 98% or greater than about 99% sequence identity with the amino acid sequence shown in FIG. 2.

Particular amino acid sequence variants may differ from those shown in FIG. 2 by insertion, addition, substitution or deletion of 1 amino acid, 2, 3, 4, 5-10, 10-20, 20-30, 30-50, 50-100, or more than 100 amino acids. In this connection, “sequence identity” means strict amino acid identity between the sequences being compared.

The present invention also includes active portions, fragments, derivatives and functional mimetics of the pH7 nSMase polypeptides of the invention.

An “active portion” of pH7 nSMase polypeptide means a peptide which is less than the full length polypeptide, but which retains at least some of its essential biological activity, preferably SMase activity or the ability to bind SMase inhibitors, such as those disclosed in EP-A-0918750. Alternatively, smaller fragments of pH7 nSMase can act as sequestrators or competitive antagonists by interacting with other molecules such as sphingomyelin and molecules that regulate pH7 nSMase activity in the cell.

A “fragment” of the pH7 nSMase polypeptide means a stretch of amino acid residues of at least 5 contiguous amino acids, often at least 7 contiguous amino acids, typically at least 9, 11 or 13 contiguous amino acids and, most preferably, at least 20 or 30 contiguous amino acids. Fragments of the polypeptide sequence may include antigenic determinants or epitopes useful for raising antibodies to a portion of the polypeptide sequence.

A “derivative” of the pH7 nSMase polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one, two, three, five or more amino acids, without fundamentally altering the essential activity of the wild type pH7 nSMase polypeptide.

“Functional mimetic” means a substance which may not contain an active portion of the pH7 nSMase amino acid sequence, and probably is not a peptide at all, but which retains an essential biological activity of natural pH7 nSMase polypeptide. The design and screening of candidate mimetics is described in detail below.

An pH7 nSMase polypeptide according to the present invention may be isolated and/or purified (e.g. using an antibody) for instance after production by expression from encoding nucleic acid (for which see below). Polypeptides according to the present invention may also be generated wholly or partly by chemical synthesis. The isolated and/or purified polypeptide may be used in formulation of a composition, which may include at least one additional component, for example a pharmaceutical composition including a pharmaceutically acceptable excipient, vehicle or carrier. A composition including a polypeptide according to the invention may be used in prophylactic and/or therapeutic treatment as discussed below.

A polypeptide, peptide fragment, allele, mutant or variant according to the present invention may be used as an immunogen or otherwise in obtaining specific antibodies. Antibodies are useful in purification and other manipulation of polypeptides and peptides, diagnostic screening and therapeutic contexts. This is discussed further below.

A polypeptide according to the present invention may be used in screening for molecules which affect or modulate its activity or function. Such molecules may be useful in a therapeutic (possibly including prophylactic) context.

The pH7 nSMase polypeptides can also be linked to a coupling partner, e.g. an effector molecule, a label, a drug, a toxin and/or a carrier or transport molecule. Techniques for coupling the peptides of the invention to both peptidyl and non-peptidyl coupling partners are well known in the art. In one embodiment, the carrier molecule is a 16 amino acid peptide sequence derived from the homeodomain of Antennapedia (e.g. as sold under the name “Penetratin”), which can be coupled to a peptide via a terminal Cys residue. The “Penetratin” molecule and its properties are described in WO 91/18981.

pH7 nSMase Antagonists

pH7 nSMase antagonists (also referred to a inhibitors) include substances which have one or more of the following properties:

(a) substances capable of inhibiting expression of pH7 nSMase;

(b) substances capable of reducing the levels of pH7 nSMase present in a target tissue or cell type by binding to and neutralizing the pH7 nSMase; and/or,

(c) substances capable of counteracting a biological property of pH7 nSMase protein, particularly its sphingomyelinase activity.

Examples of pH7 nSMase antagonists are the nSMase inhibitors of EP-A-0918750 and antibodies capable of specifically binding to pH7 nSMase protein. Anti-pH7 nSMase antibodies and their production are discussed below.

Anti-pH7 nSMase Antibodies

A further important use of the pH7 nSMase polypeptides is in raising antibodies that have the property of specifically binding to the pH⁷ nSMase polypeptides, or to fragments or active portions thereof. Polyclonal antibodies can be raised in a rabbit or similar animal and affinity purified. The are useful tools as they can recognise pH7 nSMase epitope(s). These and other antibodies that can be made based on the disclosure herein can be used as a diagnostic tools and in the characterisation of pH7 nSMase.

It is possible to produce monoclonal antibodies to pH7 nSMase protein and the techniques for doing this are well established in the art. Monoclonal antibodies can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-239400. A hybridoma producing a monoclonal antibody may be subject to genetic mutation or other changes, which may or may not alter the binding specificity of antibodies produced.

The provision of the novel pH7 nSMase polypeptide enables for the first time the production of antibodies able to bind it specifically. Accordingly, a further aspect of the present invention provides an antibody able to bind specifically to the polypeptide whose sequence is given in FIG. 2. Such an antibody may be specific in the sense of being able to distinguish between the polypeptide it is able to bind and other human polypeptides for which it has no or substantially no binding affinity (e.g. a binding affinity of about 1000×worse). In particular, the antibody may be able to distinguish between pH7 nSMase and other, previously known, SMases (particularly pH8 nSMase). It is within the capability of the skilled person to determine antibody specificity for one particular protein compared to another specified protein. Specific antibodies bind an epitope on the molecule which is either not present or is not accessible on other molecules. Antibodies according to the present invention may be specific for the wild-type polypeptide.

Antibodies according to the invention may be specific for a particular mutant, variant, allele or derivative polypeptide as between that molecule and the wild-type pH7 nSMase polypeptide, so as to be useful in diagnostic and prognostic methods as discussed below. Antibodies are also useful in purifying the polypeptide or polypeptides to which they bind, e.g. following production by recombinant expression from encoding nucleic acid.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanised antibodies in which CDRs from a non-human source are grafted onto human framework regions, typically with the alteration of some of the framework amino acid residues, to provide antibodies which are less immunogenic than the parent non-human antibodies, are also included within the present invention.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The mode of determining binding is not a feature of the present invention and those skilled in the art are able to choose a suitable mode according to their preference and general knowledge.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid therefor. Antibodies may modulate the activity of the polypeptide to which they bind and so, if that polypeptide has a deleterious effect in an individual, may be useful in a therapeutic context (which may include prophylaxis).

An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

Diagnostic Methods

A number of methods are known in the art for analysing biological tissue samples from individuals to determine whether the tissue sampled carries a particular gene or allele. The purpose of such analysis may be used for diagnosis or prognosis, to assist a physician in determining the severity or likely course of a condition and/or to optimise treatment of it.

Such methods can be used in accordance with the present invention to detect the presence of pH7 nSMase, or of allelic variants thereof, in a tissue sample. For example, the absence of pH7 nSMase in a patient (from a tissue sample in which one would expect to detect its presence) may be indicative of a condition associated with a lack of, or reduced, SMase activity, such as immune deficiency. Conversely, excessive levels of pH7 nSMase, or the detection of pH7 nSMase in a tissue sample in which one would not normally expect to find it, may be indicative of a condition associated with excess SMase activity, such as auto-immune disorders and tumours. Furthermore, the provision of screening methods for pH7 nSMase allows the identification of conditions associated with the lack or overexpression of pH7 nSMase, and the characterisation of normal tissue distribution of pH7 nSMase.

Broadly, the methods divide into those screening for the presence or absence of pH7 nSMase nucleic acid sequences and those that rely on detecting the presence or absence of the pH7 nSMase polypeptide. The methods make use of biological samples from individuals that are suspected of contain the nucleic acid sequences or polypeptide. Examples of biological samples include blood, plasma, serum, tissue samples, tumour samples, saliva and urine.

Exemplary approaches for detecting pH7 nSMase nucleic acid or polypeptides include:

(a) comparing the sequence of nucleic acid in the sample with the pH7 nSMase nucleic acid sequence to determine whether the sample from the patient contains mutations; or,

(b) determining the presence in a sample from a patient of the pH7 nSMase polypeptide and, if present, determining whether the polypeptide is full length, and/or is mutated, and/or is expressed at the normal level; or,

(c) using DNA fingerprinting to compare the restriction pattern produced when a restriction enzyme cuts a sample of nucleic acid from the patient with the restriction pattern obtained from normal pH7 nSMase nucleic acid sequence or from known mutations thereof; or,

(d) using a specific binding member capable of binding to a pH7 nSMase nucleic acid sequence (either a normal sequence or a known mutated sequence), the specific binding member comprising nucleic acid hybridisable with the pH7 nSMase sequence, or substances comprising an antibody domain with specificity for a native or mutated pH7 nSMase nucleic acid sequence or the polypeptide encoded by it, the specific binding member being labelled so that binding of the specific binding member to its binding partner is detectable; or,

(e) using PCR involving one or more primers based on normal or mutated pH7 nSMase nucleic acid sequence to screen for normal or mutant pH7 nSMase nucleic acid in a sample from a patient.

Determination of the presence or absence of pH8 nSMase (e.g. in order to identify tissues most susceptible to pH7 nSMase inhibition) may be done in a corresponding manner.

A “specific binding pair” comprises a specific binding member (sbm) and a binding partner (bp) which have a particular specificity for each other and which in normal conditions bind to each other in preference to other molecules. Examples of specific binding pairs are antigens and antibodies, molecules and receptors and complementary nucleotide sequences. The skilled person will be able to think of many other examples and they do not need to be listed here. Further, the term “specific binding pair” is also applicable where either or both of the specific binding member and the binding partner comprise a part of a larger molecule. In embodiments in which the specific binding pair are nucleic acid sequences, they will be of a length to hybridise to each other under the conditions of the assay, preferably greater than 10 nucleotides long, more preferably greater than 15 or 20 nucleotides long.

In most embodiments for screening for the presence of variants of the pH7 nSMase nucleic acid, the pH7 nSMase nucleic acid in the sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art.

A variant form of the pH7 nSMase nucleic acid may contain one or more insertions, deletions, substitutions and/or additions of one or more nucleotides compared with the wild-type sequence which may or may not disrupt the gene function. Differences at the nucleic acid level are not necessarily reflected by a difference in the amino acid sequence of the encoded polypeptide, but may be linked to a known dysfunction. However, a mutation or other difference in a gene may result in a frame-shift or stop codon, which could seriously affect the nature of the polypeptide produced (if any), or a point mutation or gross mutational change to the encoded polypeptide, including insertion, deletion, substitution and/or addition of one or more amino acids or regions in the polypeptide. A mutation in a promoter sequence or other regulatory region may prevent or reduce expression from the gene or affect the processing or stability of the mRNA transcript.

There are various methods for determining the presence or absence in a test sample of a particular nucleic acid sequence, such as the sequence shown in FIG. 1 or a mutant, variant or allele thereof. Exemplary tests include nucleotide sequencing, hybridization using nucleic acid immobilized on chips, molecular phenotype tests, protein truncation tests (PTT), single-strand conformation polymorphism (SSCP) tests, mismatch cleavage detection and denaturing gradient gel electrophoresis (DGGE). These techniques and their advantages and disadvantages are reviewed in Nature Biotechnology, 15:422-426, 1997.

Tests may be carried out on preparations containing genomic DNA, cDNA and/or mRNA. Testing cDNA or mRNA has the advantage of the complexity of the nucleic acid being reduced by the absence of intron sequences, but the possible disadvantage of extra time and effort being required in making the preparations. RNA is more difficult to manipulate than DNA because of the wide-spread occurrence of RNAses.

Nucleic acid in a test sample may be sequenced and the sequence compared with the sequence shown in FIG. 1, to determine whether or not a difference is present.

Since it will not generally be time- or labour-efficient to sequence all nucleic acid in a test sample or even the whole pH7 nSMase coding sequence, a specific amplification reaction such as PCR using one or more pairs of primers may be employed to amplify the region of interest in the nucleic acid, for instance the region encoding the carboxy end of the pH7 nSMase protein, in which sequence differences are known to occur. The amplified nucleic acid may then be sequenced as above, and/or tested in any other way to determine the presence or absence of a particular feature. Nucleic acid for testing may be prepared from nucleic acid removed from cells or in a library using a variety of other techniques such as restriction enzyme digest and electrophoresis.

Nucleic acid may be screened using a variant- or allele-specific probe. Under suitably stringent conditions, specific hybridisation of such a probe to test nucleic acid is indicative of the presence of the sequence alteration in the test nucleic acid. For efficient screening purposes, more than one probe may be used on the same test sample.

Allele- or variant-specific oligonucleotides may similarly be used in PCR to specifically amplify particular sequences if present in a test sample. Assessment of whether a PCR band contains a gene variant may be carried out in a number of ways familiar to those skilled in the art. The PCR product may for instance be treated in a way that enables one to display the mutation or polymorphism on a denaturing polyacrylamide DNA sequencing gel, with specific bands that are linked to the gene variants being selected.

An alternative or supplement to looking for the presence of variant sequences in a test sample is to look for the presence of the normal sequence, e.g. using a suitably specific oligonucleotide probe or primer.

Use of oligonucleotide probes and primers has been discussed in more detail above.

Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The degree of base-pairing between the two molecules will be sufficient for them to anneal despite a mis-match. Various approaches are well known in the art for detecting the presence of a mis-match between two annealing nucleic acid molecules.

For instance, RNAse A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid. Other approaches rely on the use of enzymes such as resolvases or endonucleases.

Thus, an oligonucleotide probe that has the sequence of a region of the normal pH7 nSMase gene (either sense or antisense strand) in which mutations are known to occur may be annealed to test nucleic acid and the presence or absence of a mis-match determined. Detection of the presence of a mis-match may indicate the presence in the test nucleic acid of a mutation. On the other hand, an oligonucleotide probe that has the sequence of a region of the pH7 nSMase nucleic acid sequence including a mutation may be annealed to test nucleic acid and the presence or absence of a mis-match determined. The absence of a mis-match may indicate that the nucleic acid in the test sample has the normal sequence. In either case, a battery of probes to different regions of the gene may be employed.

The presence of differences in sequence of nucleic acid molecules may be detected by means of restriction enzyme digestion, such as in a method of DNA fingerprinting where the restriction pattern produced when one or more restriction enzymes are used to cut a sample of nucleic acid is compared with the pattern obtained when a sample containing the normal gene or a variant or allele is digested with the same enzyme or enzymes.

The presence or the absence of an important regulatory element in a promoter or other regulatory sequence located in introns may also be assessed by determining the level of mRNA production by transcription or the level of polypeptide production by translation from the mRNA.

A test sample of nucleic acid may be provided for example by extracting nucleic acid from cells, e.g. in saliva or preferably blood, or for pre-natal testing from the amnion, placenta or foetus itself.

There are various methods for determining the presence or absence in a test sample of a particular polypeptide, such as the polypeptide with the amino acid sequence shown in FIG. 2 or an amino acid sequence mutant, variant or allele thereof.

A sample may be tested for the presence of a binding partner for a specific binding member such as an antibody (or mixture of antibodies), specific for one or more particular variants of the polypeptide shown in FIG. 2.

A sample may be tested for the presence of a binding partner for a specific binding member such as an antibody (or mixture of antibodies), specific for the polypeptide shown in FIG. 2.

In such cases, the sample may be tested by being contacted with a specific binding member such as an antibody under appropriate conditions for specific binding, before binding is determined, for instance using a reporter system as discussed. Where a panel of antibodies is used, different reporting labels may be employed for each antibody so that binding of each can be determined.

A specific binding member such as an antibody may be used to isolate and/or purify its binding partner polypeptide from a test sample, to allow for sequence and/or biochemical analysis of the polypeptide to determine whether it has the sequence and/or properties of the polypeptide whose sequence is shown in FIG. 2, or if it is a mutant or variant form. Amino acid sequence is routine in the art using automated sequencing machines.

Therapeutics

Pharmaceuticals and Peptide Therapies

pH7 nSMase polypeptides and antagonists and agonists may be useful in the treatment of a wide range of disorders because of the biological activities of pH7 nSMase. Broadly the conditions that can be treated fall into the areas of tumours (particularly solid tumours), inflammatory disease and auto-immune disease.

The pH7 nSMase polypeptides, antagonists (e.g. antibodies), peptides, nucleic acids and vectors of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection.

Preservatives, stabilisers, buffers, antioxidants and/or other additives may be included, as required.

Whether it is a polypeptide, antibody, peptide, nucleic acid molecule, small molecule or other pharmaceutically useful compound according to the present invention that is to be given to an individual, administration is preferably in a prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980 Suitable doses for the guanidine derivatives of EP-A-0918750 are provided therein.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, eg in a viral vector (a variant of the VDEPT technique—see below). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WO90/07936).

A composition may be administered alone or in combination with other treatments, either simultaneously or sequentially, dependent upon the condition to be treated.

Methods of Gene Therapy

As a further alternative, the nucleic acid encoding the authentic biologically active pH7 nSMase polypeptide could be used in a method of gene therapy, to treat a patient who is unable to synthesize the active polypeptide or unable to synthesize it at the normal level, thereby providing the effect provided by wild-type pH7 nSMase.

Vectors such as viral vectors have been used in the prior art to introduce genes into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted tumour cells, providing long lasting effect, or alternatively the treatment may have to be repeated periodically.

A variety of vectors, both viral vectors and plasmid vectors, are known in the art, see U.S. Pat. No. 5,252,479 and WO93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

As mentioned above, the aim of gene therapy using nucleic acid encoding the pH7 nSMase polypeptide, or an active portion thereof, is to increase the amount of the expression product of the nucleic acid in cells in which the level of the wild-type pH7 nSMase polypeptide is absent or present only at reduced levels. Target cells for gene therapy include cell types of the immune system, bone-marrow cells and tumour cells. Cell engineering can be used to provide the overexpression or repression of pH7 nSMase in transfected cell lines which can then be subsequently transplanted to humans. Gene therapy can be employed using a promoter to drive pH7 nSMase expression in a tissue specific manner (e.g. an insulin promoter linked to pH7 nSMase cDNA will overexpress pH7 nSMase in β-cells and transiently in the brain). If defective function of pH7 nSMase is involved in neurological disease, pH7 nSMase can be overexpressed in transformed cell lines for transplantation.

Gene transfer techniques which selectively target the pH7 nSMase nucleic acid to target tissues are preferred. Examples of this included receptor-mediated gene transfer, in which the nucleic acid is linked to a protein ligand via polylysine, with the ligand being specific for a receptor present on the surface of the target cells.

Antisense technology based on the pH7 nSMase nucleic acid sequences is discussed above.

Methods of Screening for Drugs

A polypeptide according to the present invention may be used in screening for molecules which affect or modulate its activity or function. Such molecules may be useful in a therapeutic (possibly including prophylactic) context.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility.

A method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Combinatorial library technology provides an efficient way of testing a potentially vast number of different substances for ability to modulate activity of a polypeptide. Such libraries and their use are known in the art. The use of peptide libraries is preferred.

Prior to or as well as being screened for modulation of activity, test substances may be screened for ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide. Alternatively, the screen could be used to screen test substances for binding to a pH7 nSMase specific binding partner, to find mimetics of the pH7 nSMase polypeptide, e.g. for testing as therapeutics.

Following identification of a substance which modulates or affects polypeptide activity, the substance may be investigated further. Furthermore, it may be manufactured and/or used in preparation, i.e. manufacture or formulation, of a composition such as a medicament, pharmaceutical composition or drug. These may be administered to individuals.

Thus, the present invention extends in various aspects not only to a substance identified using a nucleic acid molecule as a modulator of polypeptide activity, in accordance with what is disclosed herein, but also a pharmaceutical composition, medicament, drug or other composition comprising such a substance, a method comprising administration of such a composition to a patient, e.g. for treatment (which may include preventative treatment) of a condition, use of such a substance in manufacture of a composition for administration, and a method of making a pharmaceutical composition comprising admixing such a substance with a pharmaceutically acceptable excipient, vehicle or carrier, and optionally other ingredients.

A substance identified using as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. Alanine scans of peptide are commonly used to refine such peptide motifs. These parts or residues constituting the active region of the compound are known as its “pharmacophore”.

Once the pharmacophore has been found, its structure is modelled to according its physical properties, eg stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. Alternatively, where the mimetic is peptide based, further stability can be achieved by cyclising the peptide, increasing its rigidity. The mimetic or mimetics found by this approach can then be screened to see whether they have the target property, or to what extent they exhibit it. Further optimisation or modification can then be carried out to arrive at one or more final mimetics for in vivo or clinical testing.

Similarly, optimisation or modification may be carried out on any potentially pharmaceutically active substance identified herein (such as nSMase inhibitors) to arrive at compounds for in vivo or clinical testing.

As used herein “percent (%) amino acid sequence identity” of a candidate sequence to a reference sequence is defined as the percentage of amino acid residues in the candidate sequence that are identical with the amino acid residues in the reference sequence, after aligning the sequences and introducing gaps, if necessary, to achieve the maximum percent sequence identity, and not considering any conservative substitutions as part of the sequence identity. The % identity values used herein are generated by WU-BLAST-2 which was obtained from [Altschul et al., Methods in Enzymology, 266:460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSPS2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region, multiplied by 100. The “longer” sequence is the one having the most actual residues in the aligned region (gaps introduced by WU-Blast-2 to maximize the alignment score are ignored).

In a similar manner, “percent (%) nucleic acid sequence identity” of a candidate sequence to a reference sequence is defined as the percentage of nucleotide residues in the candidate sequence that are identical with the nucleotide residues in the reference sequence. The identity values used herein were generated by the BLASTN module of WU BLAST-2 set to the default parameters, with overlap span and overlap fraction set to 1 and 0.125, respectively.

As will be clear from the foregoing disclosure, the reference sequence need not be an entire nucleic or amino acid sequence as shown in FIG. 1 or FIG. 2, but may for example be a portion thereof.

pH8 nSMase

From the information provided herein, particularly in examples 1 and 2, it will be possible to isolate the pH8 nSMase protein (see example 1) from fractions containing it (see example 2), digest it, and sequence one or more resultant polypeptide or oligopeptide fragments. From this, it may be possible (if pH8 nSMase has a cDNA sequence similar to that of other known cDNA sequences) to use an analogous procedure to that described in example 1 to clone the pH8 nSMase gene or coding sequence. Alternatively, the sequence of one or more oligopeptidse or polypeptides could be used to produce degenerate oligonucleotide sequences, which can be used to probe a cDNA library (preferably one prepared from cells shown herein to possess pH8 nSMase activity), to identify clones encoding pH8 nSMase. Positive clones may then be sequenced and the process can be repeated using probes based on the positive clones until the full length coding sequence is obtained. The encoded amino acid sequence may the be deduced.

An alternative and preferred cloning strategy for pH8 nSMase is as follows. It is known from the teaching herein that the pH8 form is expressed at high levels in brain tissue. Jurkat cells (which do not express the pH8 form) are transfected with vectors from a human brain cDNA library. Cells expressing pH8 nSMase are selected by their resistance to anti-APO-1 antibody-induced apoptosis in the presence of sphingomyelinase inhibitor (e.g. C11AG); control cells (lacking pH8 nSMase) die. The inserted cDNA sequence is amplified from selected cells expressing pH nSMase using 5′ and 3′ cloning primers (based on the cloning vector) to yield the pH8 nSMase coding sequence, from which the deduced amino acid sequence is deduced.

Accordingly, in a further aspect, the present invention provides a nucleic acid molecule comprising a nucleic acid sequence encoding pH8 nSMase and fragments thereof which differ from the coding sequences of aSMase and pH7 nSMase. In a still further aspect, the present invention provides a polypeptide comprising the deduced amino acid sequence of pH8 nSMase and fragments thereof which differ from the amino acid sequences of aSMase and pH7 nSMase. The nucleic acid molecule, fragments thereof, polypeptide and fragments thereof may for example be as obtainable according to any of the above-described methods.

The following examples are provided for the better understanding of the invention in its various aspects.

### EXAMPLE 1

### Cloning pH7 nSMase

Purification of pH7 nSMase from porcine liver

Porcine liver (10 kg) was minced and homogenised in 50 mM Tris pH7.5, 1 mM NaF, 1 mM NaVO3. After centrifugation at 100 g for 10 minutes the supernatant was centrifuged for 25 minutes at 6000 g. The pellet was suspended in 0.1% Triton X 100 followed by centrifugation at 20000 g for 20 minutes.

Proteins were precipitated from the supernatant by the addition of ammonium sulphate. Proteins precipitating between 30% and 50% saturation were fractionated by iso-propanol precipitation. Proteins precipitating between 20 and 40% iso-propanol were separated by FPLC on a Mono-Q column. Fractions containing nSMAse activity contained a single portion of an apparent size of 44 kD.

Identification of DNA Sequence of pH7 nSMase Gene

The purified protein was digested with trypsin and the resulting peptides were fractionated by HPLC. The sequence of one peptide could be determined: RIVVLNTNLYY.

In a gene bank search a single cDNA sequence (ASML gene, accession number: Y08134) was found. A function has not been described for this gene.

Cloning and Expression of pH7 nSMase

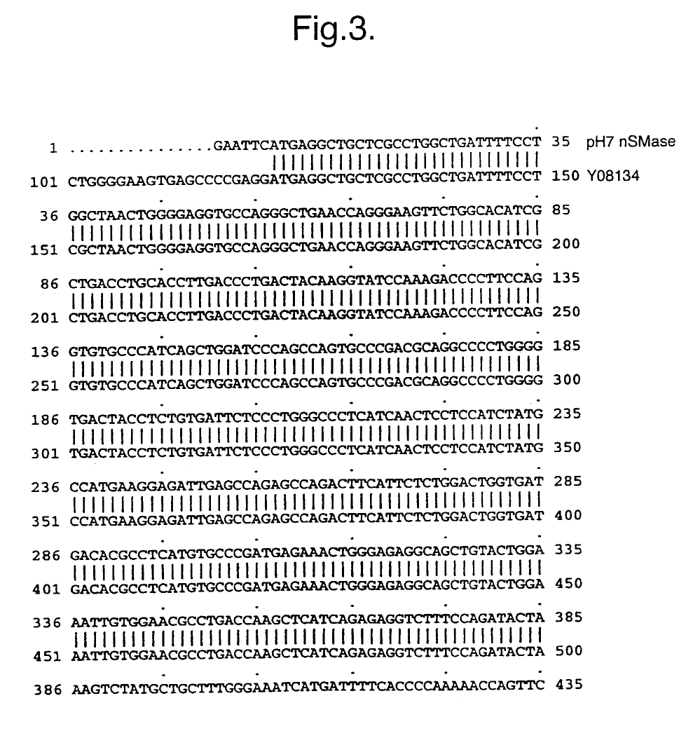

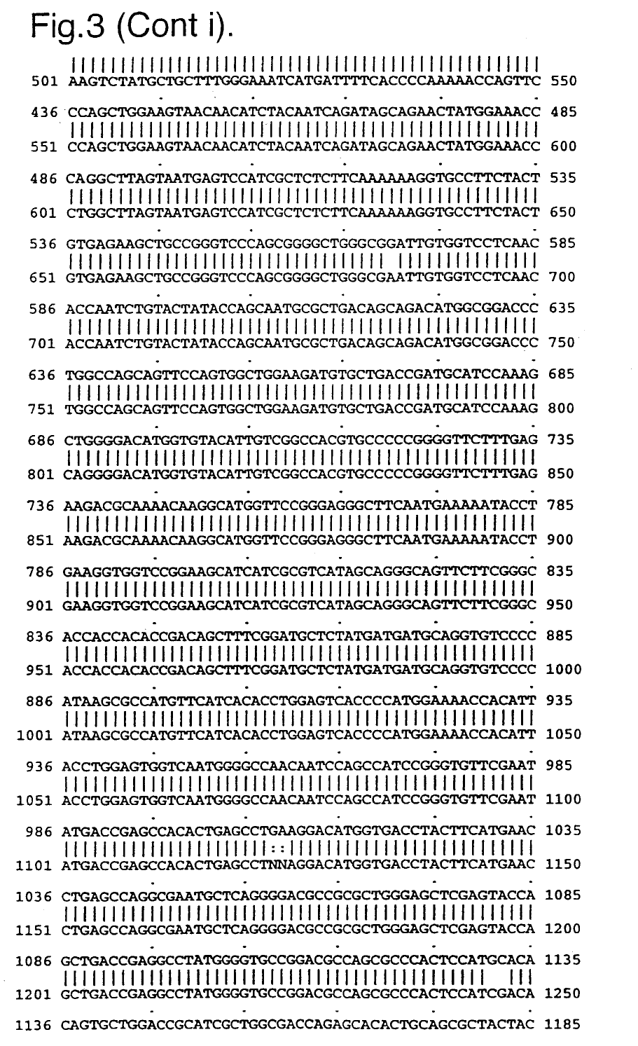

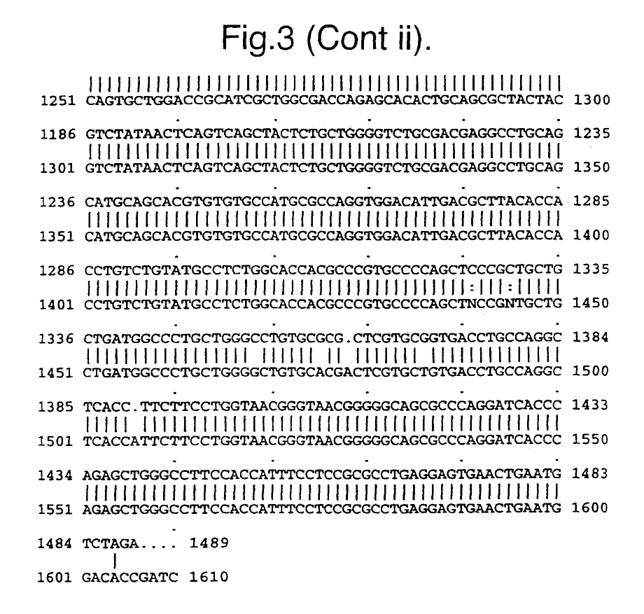

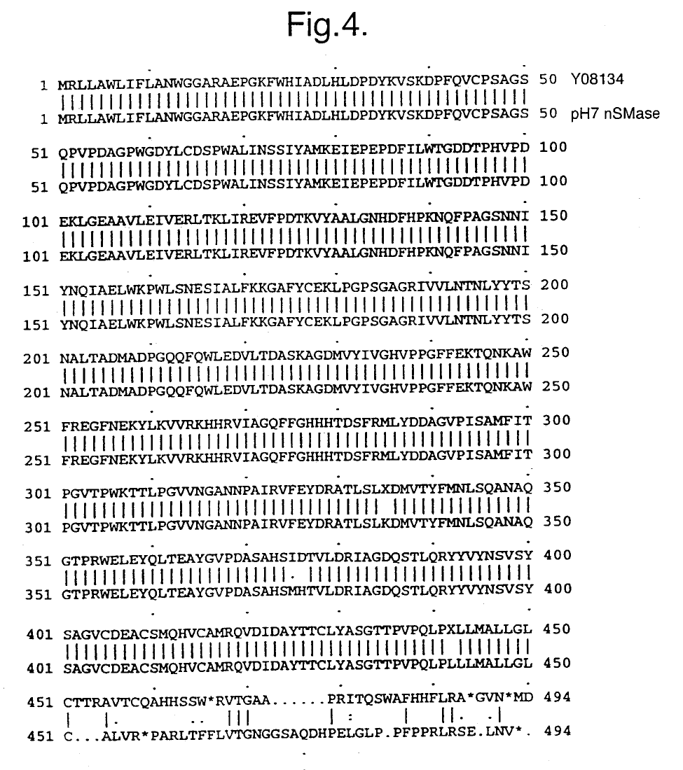

Primers for the 5′ and 3′ end of ASML were constructed and a cDNA from the Jurkat human T cell line was amplified. This cDNA was sequenced and several differences were found when the sequence was compared with the known sequence of ASML (FIG. 3), as were differences in deduced amino acid sequence (FIG. 4).

The cDNA was cloned onto two different eukaryotic expression systems (pCDNA3 and pKEX). In addition a 400 base fragment of the newly cloned (nSMase) gene was inserted in opposite orientation (anti-sense). After transfection in human Jurkat cells, clones being resistant to G418 or Hygromycin were selected. Selected populations were assayed for nSMase activity. In cells transfected with nSMase gene a 5 fold higher activity was found. In clone 4/B2 transfected with the anti-sense construct the nSMase activity was reduced by 50% (Table 1).

TABLE 1
pH 7 nSMase activity in transfected Jurkat cells.

| | Transferred gene | PCH counts |
|---|---|---|
| J16 control | pCDNA3 | 385 |
| clone 4/F12 | pH7 nSMase/pKEX2XR | 1380 |
| uncloned pH7 nSMase | pH7 nSMase/pCDNA3 | 1997 |
| population | | |
| clone 4B12 | anti-sense pH7 | 172 |
| | nSMase/pKEX2XR | |

Membranes were isolated from cells by centrifugation at 3000 g for 5 minutes after 10 seconds sonification in 20 mM TRIS pH 7.0 and nSMase was eluted with 0.1 Triton X 100. From each preparation 5 μg of protein were used for nSMase assay in 50 μl 50 mM Tris pH7.0, 5 mM MgCl-1 mM NaF, 1 mM NaVO₃, 0.1% Triton X 100, 20 nCi ¹⁴C sphingomyelin. After 30 minutes phosphorylcholine (PCH) was extracted by the addition of 2 volumes methanol/chloroform (2/1). PCH counts were determined in an aliquot of 10 μl after separation on TLC plates (Merck 60, F254, running solvent: 0.9% NaCl/methanol/NH₄OH (50/70/5, V/V/V) in a Digital Autoradiograph.

### EXAMPLE 2

### Determination of pH Dependence of nSMase Activities

Membranes were isolated from 4/F12 cells (which overexpress pH7 nSMase, see example 1) by centrifugation at 3000 g for 5 minutes after 10 seconds sonification in 20 mM TRIS pH7.0 and nSMase was eluted with 0.1% Triton X 100.

Membrane extracts from mouse brains (0.1% Triton, according to the procedure of Gatt for extracting nSMase: Gatt, S., Biochem. Biophys. Res. Commun. 68, 235-241, 1976) were applied to a FPLC-column (Mono-Q) and proteins were eluted by increasing NaCl concentrations. Fractions were assayed for nSMase activity at pH6.5 and pH8.5. Fractions containing nSMase activity exclusively at pH8.5 were pooled.

From each preparation 5 μg of protein were used for nSMase assay in 50 μl 50 mM Tris (pH6.0, 6.5, 7.0, 7.5, 8.0, or 8.5), 5 mM MgCl₂, 1 mM NaF, 1 mM NaVO₃, 0.1% Triton X 100, 20 nCi ¹⁴C sphingomyelin. After 30 minutes phosphorylcholine (PCH) was extracted by the addition of 2 volumes methanol/chloroform (2/1). PCH counts were determined in an aliquot of 10 μl after separation on TLC plates (Merck 60, F254, running solvent: 0.9% NaCl/methanol/NH₄OH (50/70/5, V/V/V) in a Digital Autoradiograph.

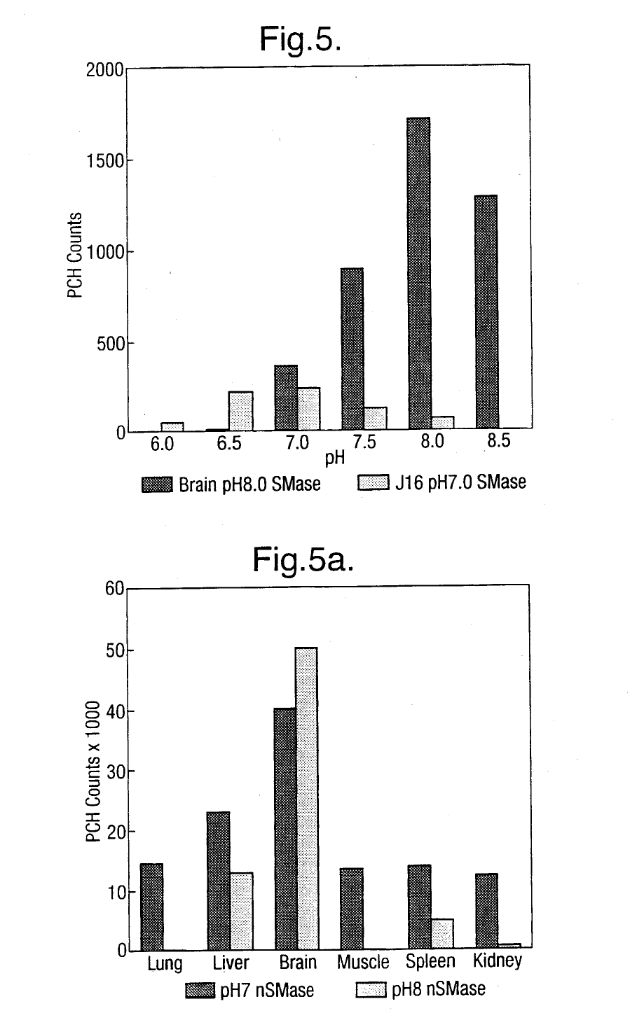

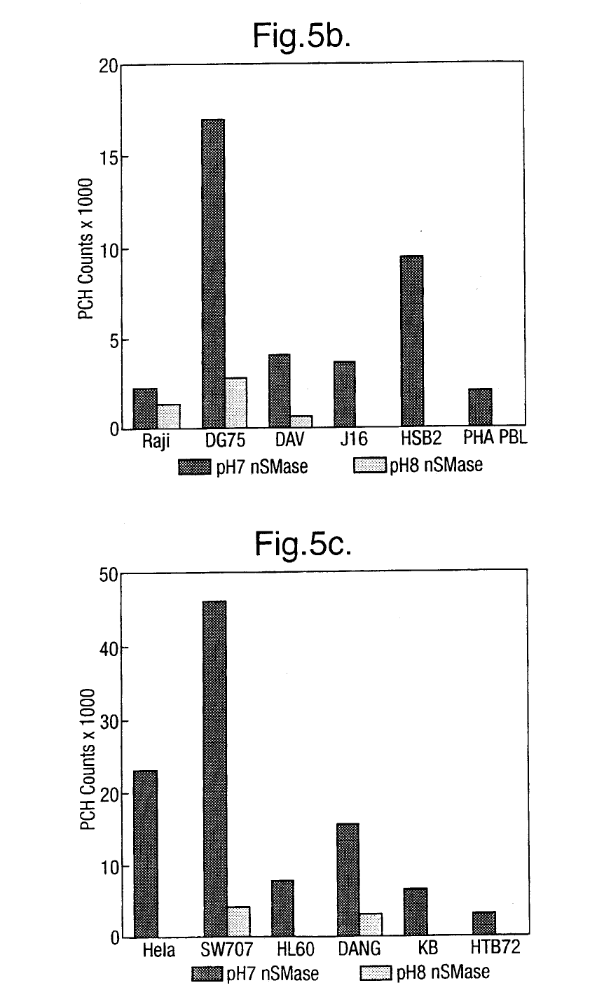

The results are shown in FIG. 5. It can be seen that there are two distinct activities with different pH optima (at pHs 7.0 and 8.0). pH7 nSMase is inactive at pH8.5 and active at pH6.5; pH8 nSMase is inactive at pH6.5 and active at pH8.5. Furthermore the two activites could be separated on anion exchange chromatography columns (data not shown). The sources of the two activities are cells known to be deficient in acid SMase (data not shown).

### EXAMPLE 3

### Determination of pH7 and pH8 nSMase Activity

Membranes were isolated from cells or tissue by centrifugation at 3000 g for 5 minutes after 10 seconds sonification in 20 mM TRIS pH7.0 and nSMase was eluted with 0.1 Triton X 100. From each preparation 5 μg of protein were used for (1) a pH7 nSMase assay (in

50 μl 50 mM Tris pH6.5, 5 mM MgCl₂, 1 mM NaF, 1 mM NaVO₃, 0.1% Triton X 100, 20 nCi ¹⁴C sphingomyelin) and (2) a pH8 nSMase assay (in 50 μl 50 mM Tris pH8.5, 5 mM MgCl₂, 1 mM NaF, 1 mM NaVO₃, 0.1% Triton X 100, 20 nCi ¹⁴C sphingomyelin).

After 30 minutes phosphorylcholine (PCH) was extracted by the addition of 2 volumes methanol/chloroform (2/1). PCH counts were determined in an aliquot of 10 μl after separation on TLC plates (Merck 60, F254, running solvent: 0.9% NaCl/methanol/NH₄OH (50/70/5, V/V/V) in a Digital Autoradiograph.

Results are shown in FIGS. 5a-5d. Different tissues and organs show different distributions of pH7 and pH8 nSMase. pH7 is present in all samples tested; pH8 is absent from certain samples, particularly tumour and lymphoma cells.

### EXAMPLE 4

### Determination of Sensitivity to SMase Inhibitor C11AG

SMase activities were determined in the presence of 0, 1, 10, 100 or 1000 μg/ml C11AG. C11AG-acetate was used as a stock solution of 10 mg/ml in water.

For pH7 nSMase assays, membranes from Jurkat cells (human T-cell lymphoma) were used.

For pH8 nSMase assays, membranes from KE37 cells (human B-cell lymphoma) were used.

For acidic SMase assays, microcosms from Jurkat cells (human T-cell lymphoma) were used.

From each preparation 5 μg of protein were used for (1) a pH7 nSMase assay (in 50 μl 50 mM Tris pH6.5, 5 mM MgCl₂, 1 mM NaF, 1 mM NaVO₃, 0.1% Triton X 100, 20 nCi ¹⁴C sphingomyelin), (2) a pH8 nSMase assay (in 50 μl 50 mM Tris pH8.5, 5 mM MgCl₂, 1 mM NaF, 1 mM NaVO₃, 0.1% Triton X 100, 20 nCi ¹⁴C sphingomyelin) and (3) an acidic SMase assay (in 50 μl 50 mM NaH₂PO₄ pH5.0, 5 mM MgCl₂, 1 mM NaF, 1 mM NaVO₃, 0.1% Triton X 100, 20 nCi ¹⁴C sphingomyelin). (In pH gradient experiments similar to that described in example, it was previously found that aSMase is inactive at pH6.5, but active at pH5.0)

After 30 minutes phosphorylcholine (PCH) was extracted by the addition of 2 volumes methanol/chloroform (2/1). PCH counts were determined in an aliquot of 10 μl after separation on TLC plates (Merck 60, F254, running solvent: 0.9% NaCl/methanol/NH₄OH (50/70/5, V/V/V) in a Digital Autoradiograph. PCH counts of the uninhibited reaction were set 100% enzyme activity. Each assay was done in quadruplicate.

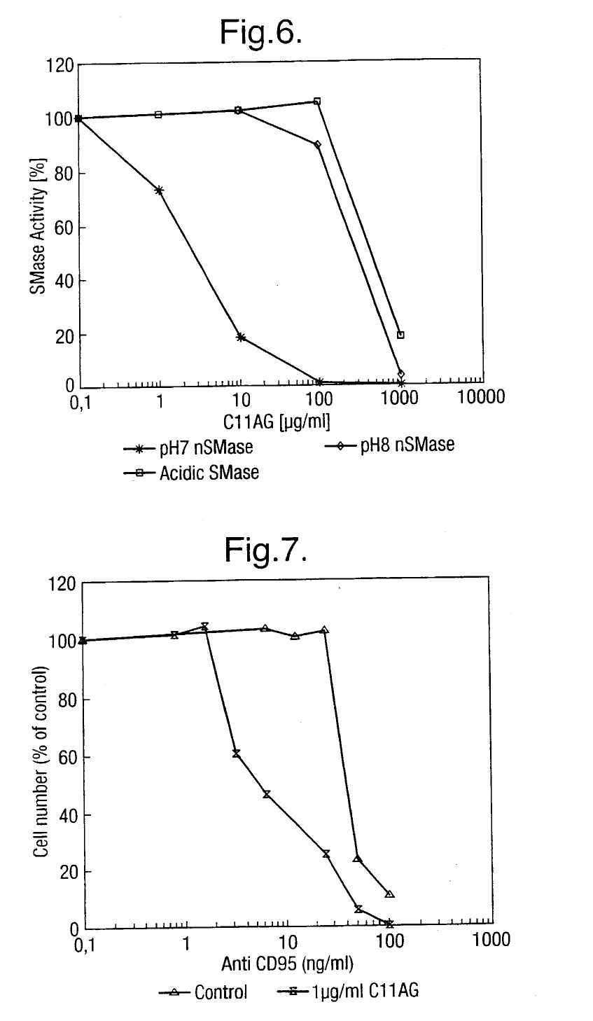

Results are shown in FIG. 6. C11AG is a much more potent inhibitor of pH7 nSMase than of pH8 nSMase or aSMase, requiring approximately 1 μg/μl for 50% inhibition, compared with approximately 300 and 400 μg/μl respectively for pH8 nSMase and aSMase.

### EXAMPLE 5

### Stimulation of Apoptosis in Human T-Cell Lymphoma Cells by an Inhibitor of pH7 nSMase.

Human T-cell lymphoma cells (Jurkat) were seeded in 96-well plates at a density of 10⁵ cells per ml. An antibody directed against CD95 (anti-APO-1) was added at various concentrations with and without C11AG at 1 μg/ml. After incubation at 37° C. for 24 hours, cell numbers were determined afer vital staining. By Nicoleti staining it was confirmed that dead cells had died due to apoptosis. All assays were done in triplicate.

Results are shown in FIG. 7. Presence of C11AG stimulated induction of apoptosis by anti-APO-1.

[image]

[image]

[image]

1. An isolated polypeptide comprising the 455 amino acid sequence or the 493 amino acid sequence set out in FIG. 2.

2. An isolated polypeptide including an amino acid sequence which has at least 80% amino acid sequence identity with the 455 amino acid sequence set out in FIG. 2, and which is identical to FIG. 2 at positions corresponding to one or more of positions 335, 375, 376, 442 and 452-455.

3. An isolated polypeptide including an amino acid sequence which has at least 80% amino acid sequence identity with the 493 amino acid sequence set out in FIG. 2, and which is identical to FIG. 2 at positions corresponding to one or more of positions 335, 375, 376, 442, 452-464, 468-482, 484-486, 489-491 and 493.

4. An isolated polypeptide according to claim 2 or claim 3 having a sequence which is identical to the sequence of FIG. 2 in at least three of said positions.

5. An isolated polypeptide according to claim 2 or 3 having a sequence which is identical to the sequence of FIG. 2 in at least five of said positions.

6. An isolated polypeptide according to claim 2 or claim 3 having a sequence which is identical to the sequence of FIG. 2 in at least seven of said positions.

7. An isolated polypeptide according to claim 3 having a sequence which is identical to the sequence of FIG. 2 in at least ten of said positions.

8. An isolated polypeptide according to any preceding claim having SMase activity.

9. An isolated polypeptide according to claim 8, wherein the SMase activity is greater at pH7 than at pH8.

10. An isolated polypeptide according to claim 9 wherein the SMase activity is greatest at about pH7.

11. An isolated polypeptide according to claim 10 wherein the SMase activity is greatest between pH6.5 and pH7.5.

12. A fragment of a polypeptide according to any preceding claim having at least 5 amino acids, which shows at least 80% sequence identity with a portion of corresponding length of the amino acid sequence of FIG. 2 and is identical to FIG. 2 at positions corresponding to one or more of the positions listed in claim 2 or claim 3.

13. A fragment according to claim 12 which has identity with some or all of residues 452-455 or residues 452-493 of FIG. 2.

14. A polypeptide or fragment according to any preceding claim wherein the level of sequence identity with the sequence of FIG. 2 is at least 90%.

15. An isolated polypeptide having SMase activity which is greater at pH8 than at pH7 or pH7.5.

16. An isolated polypeptide according to claim 15 wherein the SMase activity is greatest at a pH above 7.5, preferably above pH7.6, 7.7, 7.8 or 7.9.

17. An isolated polypeptide according to claim 16, wherein the SMase activity is greatest at about pH8.

18. An isolated polypeptide according to any one of claims 15 to 17 wherein the SMase activity is greatest between pH7.6, pH7.7, pH7.8 or pH7.9 and pH8.5.

19. An isolated polypeptide according to any one of claims 15 to 18 as obtained by, or as obtainable by, isolating SMase activity from a B cell lymphoma cell.

20. A unique fragment of a polypeptide as defined in any one of claims 15 to 19.

21. A method of identifying inhibitors or binding partners of nSMase, or of optimising previously identified such inhibitors or binding partners, the method comprising determining whether a candidate inhibitor/binding partner possesses the ability, or improved ability relative to a reference inhibitor/binding partner, to inhibit sphingomyelinase activity of, or to bind to, a polypeptide or fragment as defined in any preceding claim.

22. A method according to claim 21, wherein binding is assayed by labelling the polypeptide or fragment and determining the presence of label bound to an immobilised candidate molecule.

23. A method comprising, having previously identified or optimised an inhibitor or binding partner according to the method of claim 21 or claim 22 and optionally having previously further modified and/or optimised said inhibitor or binding partner, the step of formulating said inhibitor or binding partner as a medicament.

24. A method comprising, having formulated a medicament according to claim 23, administering said medicament to a patient having a condition treatable by inhibition of nSMase.

25. A method according to claim 24 wherein the condition is an auto-immune disorder, inflammatory disease, lymphoma or tumour.

26. A method according to claim 25 wherein the condition is an auto-immune disorder, inflammatory disease, T cell lymphoma or tumour and the inhibitor was identified and/or optimised using a polypeptide or fragment according to any one of claims 1 to 14.

27. A method according to claim 25 wherein the condition is a B cell lymphoma and the inhibitor was identified and/or optimised using a polypeptide or fragment according to any one of claims 15 to 20.

28. A nucleic acid encoding a polypeptide or fragments according to any one of claims 1 to 20.

29. A nucleic acid according to claim 28 having a nucleic acid sequence comprising nucleotides 7 to 1371 or 1485 as set out in FIG. 1.

30. A nucleic acid according to claim 28 having a nucleic acid sequence which has at least 60% sequence identity with nucleotides 7 to 1371 or 1485 of FIG. 1, or a portion thereof of corresponding length, and which encodes the same amino acid as the sequence of FIG. 1 at one or more codons corresponding to codons 335, 375, 376, 442, 452-464, 468-482, 484-486, 489-491 and 493 of FIG. 1, codon 1 corresponding to nucleotides 7 to 9.

31. A nucleic acid according to any one of claims 28 to 30 having a nucleic acid sequence which is identical to the nucleic acid sequence of FIG. 1 at one or more residues corresponding to positions 570, 1008, 1009, 1131, 1132, 1326, 1330, 1353 and 1360 and/or lacks an insert between residues corresponding to positions 1362 and 1363 and/or between residues corresponding to positions 1389 and 1390.

32. A nucleic acid according to any one of claims 28 to 31 wherein the level of identity is at least 80%.

33. An expression vector comprising a nucleic acid according to any one of claims 28 to 32 operably linked to control sequences to direct its expression in a suitable host cell.

34. An expression system transformed with a vector according to claim 33.

35. A method of producing a polypeptide or fragment according to any one of claims 1 to 20, the method comprising producing the polypeptide or fragment in an expression system according to claim 24 and isolating the polypeptide or fragment thus produced.

36. A nucleic acid having a nucleic acid sequence complementary to the nucleic acid sequence of, or capable of hybridising under physiological conditions to, a nucleic acid of any one of claims 28 to 32.

37. A transcription vector comprising a nucleic acid according to claim 36 operably linked to control sequences to direct its transcription in a suitable cell.

38. A host cell comprising a vector according to claim 37.

39. An antibody capable of specifically binding to a polypeptide or fragment according to any one of claims 1 to 20.

40. A pharmaceutical composition comprising a polypeptide, fragment, nucleic acid, vector, host cell or antibody according to any one of claims 1 to 20 and 28 to 39.

41. A polypeptide, fragment, nucleic acid, vector, host cell or antibody according to any one claims 1 to 20 and 28 to 39, for use in a method of medical treatment or diagnosis.

42. A method of assaying a test sample for likely susceptibility to nSMase inhibition, the method comprising determining in the test sample expression of pH8 nSMase, wherein reduced expression, low expression or the absence or substantial absence of expression of pH8 nSMase is indicative of likely susceptibility of the test sample to nSMase inhibition using an inhibitor of pH7 nSMase.

43. A method according to claim 42 wherein pH7 nSMase expression is also determined, expression of pH⁷ nSMase combined with reduced expression, low expression or the absence or substantial absence of expression of pH8 nSMase being indicative of likely susceptibility of the test sample to nSMase inhibition using an inhibitor of pH7 nSMase.

44. A method of selectively inhibiting nSMase in a cell or tissue having reduced expression, low expression or the absence or substantial absence of expression of pH8 nSMase, the method comprising administering to said cell or tissue a pH7 nSMase inhibitor.

45. A method of enhancing CD95-ligand/fas-mediated apoptosis in a cell, tissue or sample, the method comprising exposing the cell, tissue or sample to an nSMase inhibitor under conditions suitable for inducing CD95-ligand/fas-mediated apoptosis.

46. The use of a pH7 nSMase inhibitor in the preparation of a medicament for selectively inhibiting SMase in cells having reduced expression, low expression or the absence or substantial absence of expression of pH8 nSMase.

47. A method or use according to any one of claims 43 to 46 wherein said inhibitor of nSMase is a guanidine derivative, an antibody against or binding partner of pH7 nSMase, or a nucleic acid or vector according to claim 36 or claim 37.

48. A method or use according to claim 47 wherein said guanidine derivative is of the general formula
X—C(NH)NH₂,
wherein:
X can denote R₁, —NHR₁, —NH—NH—CHR₁R₂, —NH—N═CR₁R₂ or 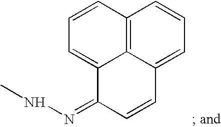
R¹ and R₂, independently of each other, can denote hydrogen, a linear or branched C₃-C₂₀ alkyl or C₃-C₂₀ cycloalkyl radical, an adamantyl, norbornyl, tricyclodecyl or benzyl radical, a pyridyl, indolyl, quinolyl, anthracenyl, phenanthryl, perinaphthyl or quinuclidinyl radical, wherein the aforementioned C₃-C₂₀ cycloalkyl radical can be substituted by a hydroxyl, a C₁-C4 alkoxy or C₁-C₄ alkyl group, a halogen atom or an amino group, and wherein if X denotes —NH—N═CR₁ R₂ only one of the substituents R₁ and R₂ can represent hydrogen,
optionally in the form of individual optical isomers, mixtures of individual isomers, or racemates, tautomers or geometric isomers, including cis/trans isomers, as well as in the form of free bases or the corresponding acid addition salts with pharmacologically acceptable acids.

49. A method or use according to claim 48 wherein X denotes —NH—NH—CH₂R₁ or —NH—N═CHR₁, and R denotes a branched or unbranched C₈-C₂₀ alkyl.

50. A method or use according to claim 49 wherein R denotes an unbranched decyl radical.

51. A method or use according to any one of claims 42 to 50 wherein said cell, tissue or sample is from an individual having or suspected to have a medical condition characterised by reduced expression, low expression or the absence or substantial absence of expression of pH8 nSMase.

52. A method or use according to any one of claims 42 to 51, wherein said cell, tissue or sample comprises an activated or autoreactive T cell, a T lymphoma cell, or a tumour cell.

53. A method or use according to claim 51 wherein said medical condition is autoimmune disease, inflammatory disease or tumour.

54. A method or use according to claim 52 or claim 53 wherein said tumour is a solid tumour.

55. A pharmaceutical composition comprising an inhibitor of nSMase and an agent capable of inducing CD95-ligand/fas-mediated apoptosis.

56. The use of an inhibitor of nSMase and an agent capable of inducing CD95-ligand/fas-mediated apoptosis in the preparation of a medicament for the treatment of autoimmune disease, inflammatory disease or tumour.

57. A composition or use according to claim 55 or claim 56, wherein said agent is an anti-fas antibody or an anti-CD95 antibody.

58. A method of inhibiting apoptosis in a cell, tissue or sample, the method comprising causing the cell, tissue or sample to express nSMase activity.

59. A method according to claim 58 comprising introducing into said cell, tissue or sample, or into an individual a nucleic acid or vector according to any one of claims 28 to 33 or a cell comprising a said nucleic acid or vector.
